(19) United States
(12) Patent Application Publication
(10) Pub. No.: US 2008/0167662 A1
(43) Pub. Date: Jul. 10, 2008

(54) Tactile feel apparatus for use with robotic operations

(76) Inventor: Anthony D. Kurtz, Saddle River, NJ (US)

Correspondence Address: The Plevy Law Firm, 10 Rutgers Place, Trenton, NJ, 08618, US

(21) Appl. No.: 11/650,643

(22) Filed: Jan. 8, 2007

(51) Int. Cl. A61B 19/00

(52) U.S. Cl. 606/130

(57) ABSTRACT

A system for providing for tactile feedback to a physician or other user employs a robotic control system, which robotic control system enables the user to control an operating instrument such as a scalpel or other instrument. Pressure transducers are placed upon the instrument and the outputs from the transducers are directed to a microprocessor and glove control circuit. The microprocessor receives the signals from the pressure transducer and the signals from the robotic control system to produce output signals for controlling a glove. The glove is worn by the physician during a robotic operation. The glove contains means on the inside of the glove, which means receive the signals generated by the microprocessor and glove control circuit and provides tactile feedback to the hand of the physician or Other user while wearing the glove. This tactile feedback provides indications to the user of the robotic control signal system as to the pressure or force applied by the surgical instrument during the robotic operation. There is also described a technique for training a physician to use this system. The system employs inflatable air sacks positioned on the inner surface of the glove but other tactile indications such as heat can be employed.

### FIELD OF THE INVENTION

This invention relates to robotic medical equipment and more particularly to apparatus for use in controlling an instrument to provide a tactile feel of the force or pressure applied by the robotic equipment.

### BACKGROUND OF THE INVENTION

Robotic control systems are widely employed in various industries. Essentially robotics is the study of problems associated with the design, application and control of robots. This includes the sensory system of the robot. The term robot has been used loosely and basically has been applied to almost any feedback controlled mechanical system. As one can ascertain, the use of robots is becoming more and more prevalent in medical procedures. Some robots have very simple mechanical designs involving only a few degrees of freedom of movement. However, the design of robot manipulators especially for medical procedures can be quite complex. For example, in a typical robot arm, six degrees of freedom of movement are required to approach an object from any orientation. There can be more degrees of movement depending upon the paths that the surgical tool, which is part of the robotic system is controlled. In regard to medical robots employed in medical procedures, certain of these have enhanced ability to manipulate tissues and to do other things required during the surgical procedure. See for example U.S. Pat. No. 6,879,880, issued on Apr. 12, 2005, to William C. Nowlin et al., and entitled “Grip Strength With Tactile Feedback For Robotic Surgery”. This patent is assigned to Intuitive Surgical Inc. As one can ascertain from that patent, systems have been employed utilizing robots for surgery and such systems show tremendous promise for increasing the number and types of surgeries which may be performed in a minimal invasive manner. The patent recognizes the fact that although force feedback systems for robotic surgery have been proposed, these are done at extreme complexity and cost. Based on the complexity and cost, such systems have not been truly implemented. Such systems increase a surgeon's dexterity and effectiveness during a surgical procedure. In prior art robotic systems the image of the surgical site is displayed adjacent control input devices. The system operator, as for example, a physician, manually manipulates the input devices thereby controlling the motion of the surgical instrument. A servo-mechanism generally moves the surgical device or tool in response to the operator's manipulation of the input devices. The motions provide translation, rotation and other actuation modes. As the servo-mechanism moves the surgical tool, the system operator retains control over the procedure. The servo-mechanism moves the surgical device to desired positions and orientations. A processor or other control device transforms the inputs from the system operator so that the tool or device movements as displayed follow the position of the input devices as perceived by the system operator. This is one way of providing feedback to an operator of a medical robotic system. This prior art feedback is a visual feedback. In the above-noted '880 patent, surgical robots and other robotic systems have enhanced grip actuation for manipulating tissues and objects with small sizes. This patent employs a master/slave system in which an error signal or gain is altered when the grip members are near a closed configuration. In regard to other robotic techniques, reference is also made to U.S. Pat. No. 7,118,582 issued on Oct. 10, 2006, entitled “Method and Apparatus for Performing Minimally Invasive Cardiac Procedures” by Y. Wang et al., and assigned to Computer Motion Inc. See also U.S. Pat. No. 7,042,184, entitled “Micro-Robot for Surgical Applications” issued on May 9, 2006, to D. Oleynikov et al., and assigned to the University of Nebraska. As one will ascertain, there are many techniques for performing medical operations using robots. These techniques can employ any type of instrument for robotic use.

The present invention provides a tactile feel to the operator of the robotic system. The operator receives a force or pressure which is felt by the operator. This force is indicative of the force or pressure applied by the surgical instrument used in the robotic procedure. In this manner, the operator has a feel for the pressure applied by the surgical instrument to optimize the control of the instrument. This tactile feel is similar to the feel experienced by the surgeon's hands during an actual operation where a robotic system is not being employed.

### SUMMARY OF THE INVENTION

A robotic system for use in controlling an instrument, by providing control signals to a mechanism for moving the instrument in various planes to accomplish a given procedure, comprising: a plurality of sensors mounted about various regions of said instrument, each sensor capable of providing an output signal proportional to a pressure applied at the region, a glove for accommodating a user's hand, the glove having tactile providing means located on the inside of the glove at given finger areas, the tactile means responsive to input signals to cause the means to provide corresponding tactile outputs and means for applying the sensor output signals to the tactile means, whereby when the glove accommodates a user's hand the user feels the tactile signals.

### BRIEF DESCRIPTION OF THE FIGURES

FIG. 1 is a schematic view of a robotic control system using tactile feedback according to this invention.

FIG. 2 is a diagram depicting a surgeon's hand holding a cutting instrument necessary to explain operation of this invention.

FIG. 3 is a partial cross-sectional and schematic view of a glove finger having tactile feedback operation according to this invention.

FIG. 4 is a schematic diagram of a pressure sensor according to this invention.

FIG. 5 consists of FIGS. 5A and 5B showing a training surgical instrument.

FIG. 6 depicts a processing system utilized for different surgeons for training according to this invention.

FIG. 7 is a schematic view of a glove having tactile feedback areas placed according to this invention.

FIG. 8 is a schematic view depicting an alternate embodiment of operating a pressure source according to this invention.

FIG. 9 is a schematic view showing a system according to this invention using a different surgical tool.

### DETAILED DESCRIPTION OF THE INVENTION

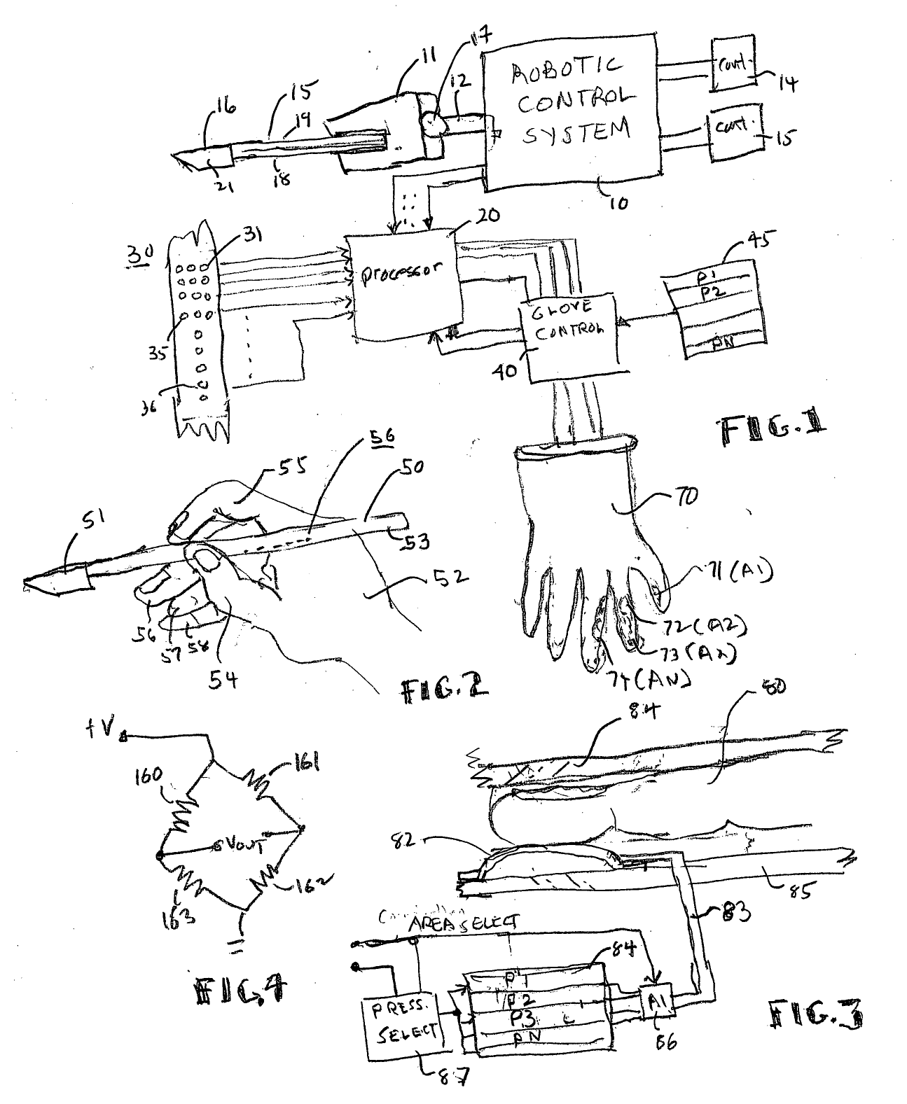

Referring to FIG. 1 there is a shown a robotic control system 10 employing a tactile feel apparatus which operates in conjunction with the system. As indicated above, robotic control systems, such as system 10, are well known in the art. See for example U.S. Pat. No. 7,083,571 entitled “Medical Robotic Arm That is Attached to an Operating Table” issued on Aug. 1, 2006 to Wang et al., and assigned to Intuitive Surgical. There are many methods and techniques for controlling surgical procedures with robotic control systems as 10. As seen the robotic control system operates in conjunction with a gripping member 11, which is referred to as an arm. The term arm is used as the robotic system moves as does the arm and hand of a surgeon during an operation. The member 11 is attached to the control system by means of a shaft 12. Shaft 12 is rotatable and the gripping member 11 can perform multiple functions as it can move in various planes. The member 11 as shown in FIG. 1 is holding a scalpel or cutting member 15 having a blade portion 16. Essentially the robotic control system can move the member 11 up and down, sideways and can rotate the arm and perform various degrees of motion. The ability of such control systems to operate accordingly is shown and fully described in the above-noted patents. The operator or user of the system has input controls such as 14 or 15 which may be joysticks, handles or other control devices, which allow him to perform an operation by moving the member 11 and therefore the scalpel 15. Usually the robotic control system 10 contains a television camera or other viewing device to enable the surgeon to view the area that the scalpel blade 16 is being used. While a scalpel and scalpel blade 16 are shown, it is known that there are various other types of medical tools which are used in medical procedures which enable the surgeon to perform a full operation. For example the scalpel 15 can be replaced as other surgical instruments which can be controlled by manipulating the arm or gripper 11. The robotic control system 10 of course produces multiple signals, which signals control the arm 11 as well as the shaft 12 and therefore the instrument attached to the arm such as the scalpel 15. The robotic control system can perform the same types of motion as the human hand can perform. In order to provide such motion, robotic control systems provide various control signals which serve to operate the arm 11 as well as the shaft, as well as the ball and socket joint 17. These control signals are directed to a microprocessor 20. The control signals for example are control signals which operate the scalpel 15 in the X, Y and Z directions as well as signals which rotate it. The various signals from the various control motors are directed into the microprocessor 20. Also shown is that the scalpel 15 has a plurality or matrix of pressure transducers or sensors or stress sensors located on the top or bottom surface as well as on the side surfaces of the scalpel 15. As pressure is applied to the scalpel while performing a cutting operation, the scalpel deflects according to the amount of the applied pressure. Arrays of pressure transducers are placed and positioned on the scalpel. The arrays can be positioned on the side of the scalpel on the cutting blade on the top and bottom. Each transducer in the array provides an output signal proportional to an applied pressure. As seen, in FIG. 1, an array of pressure transducers 30 (or stress transducers) contains a plurality of such transducers as for example 31, 35 and 36. These transducers are miniature devices and the assignee herein, namely Kulite Semiconductor Products, Inc. has many patents as well as producing such transducers for commercial use. The transducers or sensors are placed upon surfaces of the cutting instrument. The arrays respond to the pressure applied to the scalpel by the robotic control system as it commences cutting during control by the surgeon. Control signals from the robotic control system are also directed to inputs of the microprocessor 20. In any event, each transducer in the array interfaces with an input of the microprocessor. This can be done by multiplexing or by scanning each input and storing the signal from each pressure transducer (or stress transducer) during a cutting procedure. These signals can be stored in the memory of the microprocessor for later processing. Techniques for applying multiple signals to a microprocessor are well known and as indicated these signals are created by scanning all the pressure transducers and then storing the output voltages in memory for later processing by the microprocessor 20. Also, as seen, the outputs from the microprocessor are directed to a glove control circuit 40. The glove control circuit essentially is a digital signal processor which takes the output signals from the microprocessor and processes these signals as will be explained. Also shown coupled to the glove control system 40 is a pressure module 45. The pressure module 45 contains a compressed gas such as air and can provide various pressure outputs to control the inflation of bladders contained within a tactile glove 70. The amount of air under pressure contained in the pressure module 45 can be multiple pressures and as will be explained, are employed to selectively inflate various inflatable areas or bladders associated with the tactile glove 70. As seen in FIG. 1, the glove 70 receives inputs from the glove control system which inputs as will be explained, are employed to inflate various inflatable areas associated with the glove. This enables a user wearing the glove to feel pressures on his hand and fingers when placed in the glove. The pressure felt is proportional to the pressure being applied by the scalpel to the patient during the operating procedure. In this manner the user gets an approximation of the pressure applied and therefore by varying the controls of the robotic control system 10, the user can manipulate the instrument as if an actual surgical procedure was being performed. Essentially, the user places his hand in glove 70. Glove 70 has positioned on the inner surface various inflatable or bladder-like areas as 71, 72, 73 and 74. The inflatable areas as indicated are merely by way of example and these are multiple inflatable areas associated with the glove 70. Essentially what occurs is that each inflatable area receives a different pressure depending upon the pressure detected or provided by the robotic system as detected by the arrays of pressure transducers positioned on the surgical instrument. In this manner, the user receives various pressures due to the inflation of the inflatable areas. These pressures are proportional to the pressure being applied by the robotic system upon the skin or upon the body of a person or object being operated on.

As seen in FIG. 2 there is shown a right hand 52 holding a scalpel 50. It is of course understood that a left handed person would accommodate or hold an instrument in a similar manner. As seen in FIG. 2, the hand 52 is holding the scalpel 50. The scalpel has a shaft 53 and a blade 51. The scalpel is held between the thumb 54 together with the adjacent finger 55, with finger 56 acting as a support. This is similar to holding a pen or pencil. It is understood that other ways of holding an instrument are applicable. This explanation is only by way of example to show system operation. The remaining fingers as 57 and 58 do not really aid in holding the scalpel. In this manner, the glove 70 will have inflatable areas corresponding to the areas on the thumb 54, finger 55, and 56 which actually exert or feel the pressure exerted by holding the shaft 50 of the scalpel 51 while cutting or operating. Thus, as seen from FIG. 1, the inflatable areas of glove 70 correspond to those areas of the hand which participate in the particular surgical procedure. Hence, area 71 of the thumb corresponds to that area contacting the shaft 53 and so on. As seen from FIG. 1 that there may be very little or no inflatable bags associated with the pinky or another finger of the user. In any event, the glove may contain hundreds of inflatable or bladder areas throughout and the areas to be inflated are selected during the operating procedure by the control signals. A pressure profile is provided by the transducer array which pressure profile is used to inflate the corresponding bladders or inflatable area on the glove. The surgeon prior to using the glove and system performs an operation on a pig or other test area using a test profile scalpel 50 as shown in FIG. 2. The surgeon or other user holds the scalpel 50 and performs various incisions with the scalpel. The pressure exerted is measured by the pressure transducer arrays 56 placed on the shaft 53 of the scalpel. These pressure transducers 56 placed on the shaft 53 of the test scalpel correspond to those pressure transducers placed upon the robotic shaft 15 of the robotic scalpel 15 of FIG. 1. Thus, one obtains a pressure profile of the pressure imposed by the surgeon's hand when he is using the test scalpel 50. Thus, the pressure profile for the thumb 54, the finger 55, and finger 56 are known. These test pressures are stored in memory in a timed sequence. The surgeon performs the test operation with the test scalpel. The pressures provided during the test procedure can be stored on a tape either analog or digital. The surgeon then puts on a test glove as 70 of FIG. 1. The test pressures are applied to the processor 20 and glove control 20 and the stored pressures are used to activate the glove so the surgeon can “feel” the pressures as applied to the glove. The glove inflates the areas as if the procedure was being implemented by the robotic system. This allows the surgeon to obtain the exact tactile feel provided by the glove during the test operation. Thus, the surgeon has an understanding of the glove performance and hence he knows what to expect tactically during a robotic operation.

In FIG. 3 there is shown the surgeon's or a person's finger 80. The finger 80 is placed in a proper finger portion of the glove 70. The glove 70 as indicated has associated finger portions, inflatable air bag areas, as area or bladder 82. The bladder 82 is coupled to a selective source of pressure 84, via a tube 83 and a valve 86. Also coupled to the pressure source 84 is a pressure select module 87. Basically, as seen, during operation the selected area of the glove receives a signal which essentially opens valve 86 for a predetermined time to allow bladder 82 to inflate to a desired amount to thereby exert a desired pressure at the tip of finger 80. The pressure source may also have selected pressure valves stored so valve 86 can be opened for the same time but a different pressure applied. Thus, for example, the area for finger 80, which is shown as the fingertip associated with inflatable bag 82, is selected to be activated by a pressure P2. Thus, pressure P2 is selected from pressure source 84 by the signal from the pressure select module 87 which obtains its control signal from the microprocessor or via the glove control circuit 40. A second signal which is shown as the area select signal operates and opens the valve 86. The pressure applied for example is P2, thus the pressure P2 will be directed via the valve 86 and via the pressure tube 83 to the inflatable area 82 which will inflate according to the pressure P2. If additional or higher pressure is applied during a subsequent procedure or subsequent time then the area select signal will remain the same and the valve 86 will again open and the new pressure applied. Prior to opening valve 86, the inflatable area can be deflated via a port in valve 86, which port then closes to allow the new pressure to inflate bladder 82. While a single inflatable bladder 82 is shown, there may be multiple smaller inflatable areas, each of which will receive a corresponding pressure in order to inflate it according to a desired input signal. It is of course understood that while a multiple pressure source 84 is shown, other techniques can be employed, as for example, the inflatable area 82 may be filled with air and inflated and then when a desired pressure is reached the operation stops. Thus, a valve can be eliminated.

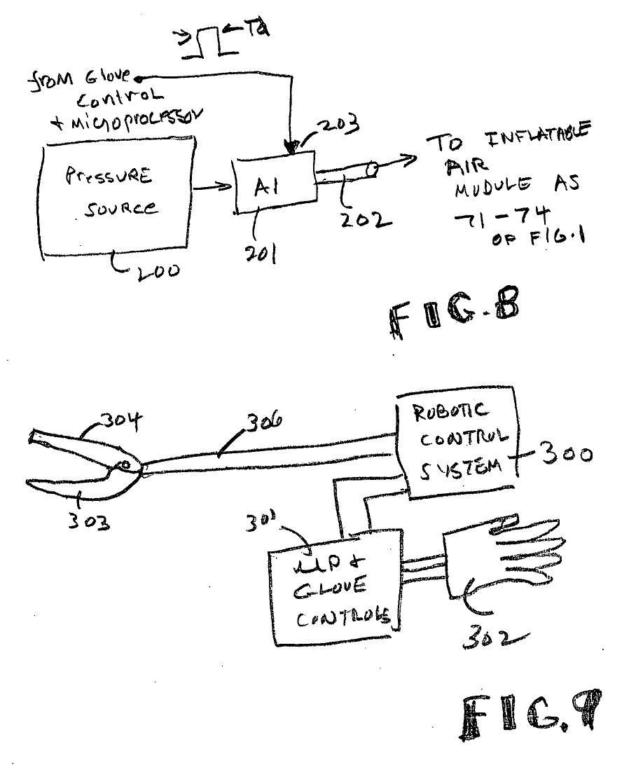

Referring to FIG. 5, there is shown a more detailed view of a test scalpel 100. The scalpel 100 has a shaft 101 and a cutting blade 102. As seen, in FIG. 5A there is shown a side view which may be the right or left side view. In any event, a plurality or array of pressure transducers is placed on each side in the holding area. The pressure transducer array for the right side would be Pₛᵣ. The left side would also have an array of pressure transducers placed thereon, which would be designated as Pₛₗ. In a similar manner, shown in 5B, the top of the scalpel shaft 101 has an array of pressure transducers designated as Pₜ for the top and array of pressure transducers designated as P_{b} for the bottom. There also may be pressure transducers as Px placed on the side of the cutting instruments and Py placed on the top of the cutting instruments. In any event, these signals are applied to the microprocessor 120. The microprocessor 120 takes the signals as emanating from the various pressure transducers through the cables which are multiple signals and scans these signals and produces a glove control profile which essentially is stored in a memory associated with the glove control module 130. As one can ascertain from the above, each surgeon, as for example surgeon A, surgeon B, and surgeon C, represented by modules 131, 132 and 133, will provide different pressures on the scalpel. Hence, each surgeon is now trained before utilizing the robotic system as briefly described above. Each surgeon takes the scalpel of FIG. 5A and actually performs a surgical procedure, which surgical procedure eventually will be performed by the robotic control system 10. This surgical procedure can be implemented on a conventional or well known dummy or on an animal. The operation can also be performed on a cadaver. In any event, the pressure profile during the entire operating procedure is monitored for that surgeon and each of the different pressures applied to the scalpel are then stored for each surgeon in the glove control memory 130. The time sequence is also stored so the pressures can be played back at the correct speed. Training may be further implemented by videotaping the surgical procedure and then playing back the video with the time sequenced pressure signals to enable the surgeon to “feel” the replayed surgical procedure. The glove 155 which is a relatively universal glove is shown in FIG. 7. Individual gloves can be provided based on the individual surgeon's pressure profile. The inflatable air bags as 150, 151 and 152 are positioned on the inner surface of the glove and are inflated and deflated according to the actual pressures on the scalpel 15 of FIG. 1. These pressures are provided during the actual operation by the robotic control system. These pressures that are received from the scalpel 15 are applied to the air bags of the glove 70 of FIG. 1 or glove 155 of FIG. 7. The glove 155 may be a different glove for each of the surgeons A, B and C, or can be a universal glove depending on the number of air pockets provided. Thus, as one can ascertain, the pressure profile of a surgeon is stored (FIG. 6) in the glove control module 130. This pressure profile then is indicative of the various pressures that the surgeon exerts on the scalpel during an operating procedure. The microprocessor 120 may use the stored glove control signals from the glove control memory 40 to alter the actual pressure signals provided during a robotic procedure. In this manner, the inflation of the air bags is a closer function of the corresponding pressure applied according to the surgeon performing the operation. It is of course understood, that the pressures generated by the robotic system as compared to the pressures generated by the actual surgery are not the same. But because the surgeon is trained during the test operation, he can respond to the generated glove pressures and act accordingly. The pressure transmitted to the surgeon will be indicative of the pressure produced by the transducer array on the scalpel 15 of FIG. 1. If the surgeon wishes to exert more or less pressure via the robotic system, he may do so by actually responding to the pressure he receives from the glove during the operating procedure. In this manner the surgeon receives actual tactile feedback of how much pressure he is applying to a given area and can therefore change the pressure. The scalpel shown in FIG. 5, as well as the glove in FIG. 7, are training mechanisms. Thus as one can ascertain, the system shown in FIG. 1 can be utilized as a training system. Once the values of the surgeon's procedures are stored, the surgeon can then proceed to operate with the robotic control system. He can also operate in a testing mode to get a feel for the pressure exerted by the glove during an operating procedure. In this manner, once he has that feel, then the necessity of comparing or manipulating the pressure signals applied to the inflatable areas would be eliminated as the surgeon would understand how the system works and understand the various pressures exerted on his hand by the system and therefore the system would not need to basically compare the pressures exerted during a training period with the pressures utilized during actual use.

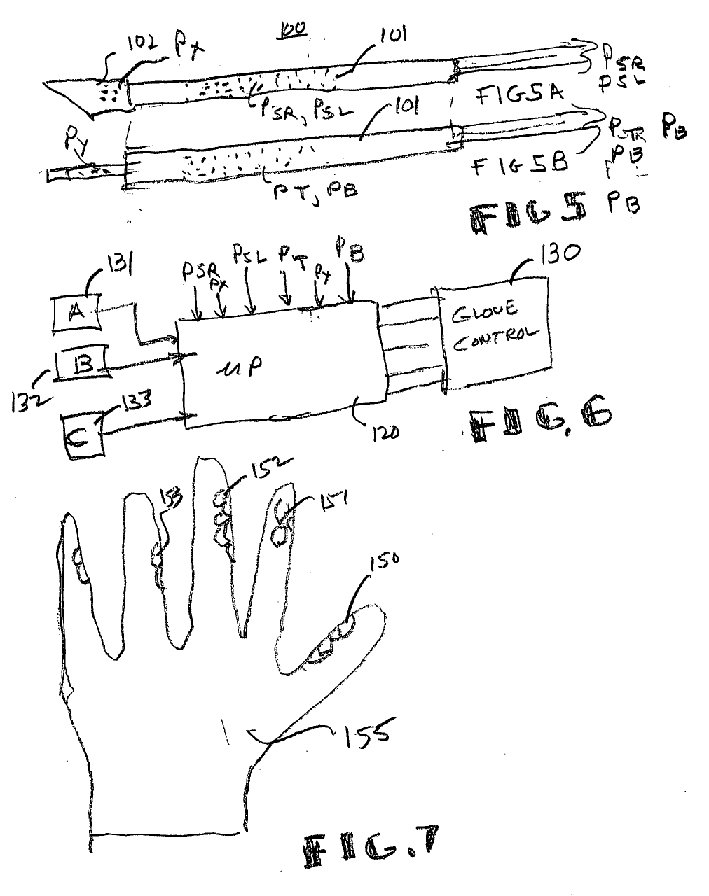

FIG. 4 of course depicts the typical pressure transducer which basically consists of a Wheatstone bridge having piezoresistive elements such as 160, 161, 162 and 163 positioned thereon. The output of the bridge is taken across the output terminals as shown. The bridge is biased with a small voltage. As indicated the pressure transducer arrays are supplied by the assignee herein, namely Kulite Semiconductor Products, Inc. These transducers are in widespread use. It is also understood that the glove has an outer material as 84 and 85, which is thicker and less flexible than the inflatable material 82. The inflatable material 82 can be a typical inflatable, a plastic or a rubber material or latex as used in balloons. The bags are inflated based on a selected pressure, and essentially the inflation of the bag changes its height to exert a pressure to the hand of the surgeon who wears the glove. For a very high pressure the bag 82 would be inflated to a greater degree by a larger pressure source, thus pushing the surgeon's finger against the top and bottom of the glove, therefore creating a greater pressure feeling. The inflating pressure may be released when the valve 86 (FIG. 3) is accessed, prior to opening the valve the pressure is discharged through an outlet and the outlet is then closed and the new pressure applied. As one can ascertain, if the air bag has a lower pressure and a higher pressure is applied, then it will inflate to the higher pressure. In the same manner, if a lower pressure is applied then the valve will dissipate the excess pressure and allow the bag to deflate to a given level. While various selectable pressures are shown, it is of course understood that a timing signal can be produced for each different pressure whereby the valve 86 can be opened for a greater or lesser duration depending upon the pressure desired. Thus, for example, instead of having various pressure sources, one can have a single pressure source of a given pressure which will inflate each of the inflatable areas according to a different time period which specifies how long the valve 86 is opened. This, of course would be a preferable way of controlling the pressure as one can therefore obtain a much greater and linear pressure response.

Referring to FIG. 8, there is shown a pressure source 200 which contains a gas or air at a given pressure. The output of the pressure source 200 is coupled to an input of valve 201. The valve 201 has a control input 203. The control input of the valve is obtained from the glove control and microprocessor and is a time duration signal of time duration Td. Td specifies how long the valve A1 will be opened. Therefore, depending on how long the valve 201 is opened, a different amount of air will be supplied to output tube 202 which is coupled to one of the inflatable air modules or air bladder, as for example 71 to 74 of FIG. 1. In this manner the module will be inflated according to the width of the time signal and thus according to the pressure specified by the system.

The invention uses a glove having inflatable areas, or air sacks, but it is understood that one can apply different means associated with a glove to provide a surgeon with control signals or tactile signals indicative of the pressure applied by the robotic system on the scalpel. These signals could be heat signals, electric signals and so on. These signals for different heating areas could be applied to the surgeon's hands whereby a warmer temperature would indicate a higher pressure and so on. Signals can also be high voltage, low current electrical signals to tingle the hand of the surgeon. It is of course understood that the training portion of the system as indicated above, may be utilized so that the stored values of the surgeon's actual pressure can then be played back through the glove to the surgeon so the surgeon can feel exactly the signals he actually produced when performing the test surgery. In this manner the surgeon can be trained to understand exactly the type of pressure applied by the glove based on the exact operation that he performed and based on the values that were previously stored by the test scalpel shown in FIG. 6. As indicated above, these signals generated during actual procedures can also be used to modify signals sent to the surgeon. It is of course understood that the system can accommodate any different surgeon, all of whom can be trained by the training procedure. Thus, there is described a tactile system which enables a surgeon to utilize a robotic system and to obtain a tactile input regarding the operation of the robotic system as manipulating and controlling a surgical instrument.

While the instrument shown above is a scalpel, it is also understood, as shown in FIG. 9 that other instruments can be employed. Thus, in FIG. 9 there is shown a typical robotic control system 300 which has of course input controls not shown. The shaft 306 is coupled to a surgical instrument having a first finger 304 pivotably coupled to a second finger 303. The surgical instrument depicted therein can perform holding operations and can be manipulated to hold objects such as tissue, suturing needles and various other medical devices. The glove control and the microprocessor 301 and various other components depicted in FIG. 1 provide the signals to the glove. The glove is worn by the system operator to provide tactile feedback to the operator.

It is understood by one skilled in the art that there may be alternate embodiments that can be implemented and as indicated above various other devices can be associated with the gloves to apply other types of signals to the hands of the surgeon during such operating procedures. All such modifications are deemed to be encompassed within the spirit and scope of the claims appended hereto.

What is claimed is:

1. A robotic system for use in controlling a surgical instrument during a surgical procedure performed by said robotic system, said robotic system producing control signals to control the motion of said instrument comprising:
a plurality of pressure transducers mounted on said surgical instrument, each capable of providing an output signal proportional to an applied pressure,
a processor for receiving said pressure output signals for processing said signals to provide processed output signals, each one indicative of a pressure output,
a glove having a plurality of tactile means located on the inside, with said first plurality of said means positioned about an area portion of the thumb location of said glove and with a second plurality positioned about a finger location area adjacent to said thumb, said means adapted to receive said processed output signals according to said area to provide a tactile signal to said thumb and finger areas according to the pressure applied to said surgical instrument.

2. The robotic system according to claim 1, wherein said means include inflatable areas positioned on the inside of said glove and located about said thumb and finger areas, and a pressure source coupled to said areas and operative to inflate aid areas in an amount proportional to said pressure applied to said surgical instrument.

3. The robotic system according to claim 1, further including a glove control system responsive to processed signals from said processor for storing said signals and comparing said stored signals to training signals for providing output signals to said tactile means.

4. The robotic system according to claim 1, wherein said tactile means are heating means responsive to said processed signals to provide predetermined heat values according to said applied pressure.

5. The robotic system according to claim 1, wherein said tactile means are electrical means responsive to said processed signals to provide predetermined electrical values according to said applied pressure.

6. The robotic system according to claim 1 coupling said robotic control signals to said processor, to enable said processor to modify said pressure output signals according to said control signals.

7. The robotic system according to claim 1, wherein said surgical instrument is a scalpel having a holding shaft coupled to a cutting blade.

8. The robotic system according to claim 7, further including a first plurality of transducers placed on a first holding area of said shaft.

9. The robotic system according to claim 8, wherein said first plurality of transducer output signals are applied to said microprocessor to cause a first plurality of first output processed signals to be provided, with said first output signals applied to means associated with a first area of said glove.

10. A method for providing tactile feedback to a user of a robotic control system of the type for robotically controlling an instrument during a procedure, comprising the steps of:
a) placing a plurality of pressure transducers on said instrument at a predetermined area, said transducers providing output signals according to pressures applied to said area,
b) processing said output signals to provide output control signals,
c) providing a glove having finger and thumb regions,
d) placing tactile indicating means inside said glove at given areas within given regions of said glove, said tactile indicating means providing a tactile output according to an applied signal,
e) applying said control signals to said tactile means to cause said given areas to provide said tactile indications whereby when said glove accommodates a hand said hand will feel said tactile indication.

11. The method according to claim 10, wherein said robotic system is a surgical system with said instrument being a surgical instrument.

12. The method according to claim 10, wherein the step of placing includes placing inflatable areas inside said glove at said areas.

13. The method according to claim 12, including inflating said inflatable areas according to said pressure output signals.

14. The method according to claim 1, wherein the step of placing a plurality of pressure transducers includes placing piezoresistive pressure transducers each of a Wheatstone bridge configuration on said instrument.

15. A robotic system for use in controlling an instrument, by providing control signals to a mechanism for moving said instrument in various planes to accomplish a given procedure, comprising:
a plurality of sensors mounted about various regions of said instrument, each sensor capable of providing an output signal proportional to a pressure applied at said region,
a glove for accommodating a user's hand, said glove having tactile providing means located on the inside of said glove at given finger areas, said tactile means responsive to input signals to cause said means to provide corresponding tactile outputs, and
means for applying said sensor output signals as input signals to said tactile means, whereby when said glove accommodates a user's hand, said user feels said tactile signals.

16. The robotic system according to claim 15, wherein said robotic system is a surgical system and said instrument is a scalpel.

17. The robotic system according to claim 15, wherein said sensors are piezoresistive pressure sensors.

18. The robotic system according to claim 15, wherein said means for applying said sensor output signals includes a processor for receiving said output signals and for providing processed output signals for controlling said tactile means.

19. The robotic system according to claim 18, wherein said tactile means are inflatable bladders positioned on the inside of said glove at said finger areas and capable of being selectively inflated according to said sensor output signal.

20. The robotic system according to claim 19, further including:
a source of pressure coupled to said bladders including controllable means for determining the amount of fluid required to inflate said bladder to a state indicative of a desired tactile state.
